(19) United States
(12) Patent Application Publication
(10) Pub. No.: US 2022/0132840 A1
(43) Pub. Date: May 5, 2022

(54) Electronic Device Sanitizer

(71) Applicant: Tina L. Hendricks, Falmouth, ME (US)

(72) Inventor: Tina L. Hendricks, Falmouth, ME (US)

(21) Appl. No.: 17/507,957

(22) Filed: Oct. 22, 2021

Related U.S. Application Data
(60) Provisional application No. 63/107,193, filed on Oct. 29, 2020.

(51) Int. Cl. A01N 31/02; A61L 2/18; A01N 25/08; A01P 1/00

(52) U.S. Cl. CPC A01N 31/02; A01P 1/00; A01N 25/08; A61L 2/18

(57) ABSTRACT

A sanitizing wipe that kills bacteria and viruses on electronic devices without damaging the device.

### FIELD OF THE INVENTION

The invention relates to sanitizers and sanitizing wipes and, in particular, wipes that may be used to sanitize electronic devices.

### DISCUSSION OF PRIOR ART

Sanitizing wipes are relatively small moistened pieces of fabric that include a substance that is intended to kill germs, viruses, and bacteria. Different types of plastics or cloths may be used as the wipe, however, roughly ninety percent of these materials are made from nonwoven fabrics made of polyester or polypropylene. Typically some combination of water and alcohol are used to saturate the wipe so that it may kill the intended targets.

Alcohol towelettes are one form of sanitizing wipes that have been used to disinfect small surfaces such as rubber stoppers of multiple-dose medication vials or vaccine bottles. Additionally, alcohol occasionally is used to disinfect external surfaces of equipment (e.g., stethoscopes, ventilators, manual ventilation bags), Cardiopulmonary resuscitation (“CPR”) manikins, ultrasound instruments or medication preparation areas.

In the healthcare setting, “alcohol” refers to two water-soluble chemical compounds—ethyl alcohol and isopropyl alcohol—that have generally underrated germicidal characteristics. These alcohols are rapidly bactericidal rather than bacteriostatic against vegetative forms of bacteria; they also are tuberculocidal, fungicidal, and virucidal but do not destroy bacterial spores. Their cidal activity drops sharply when diluted below 50% concentration, and the optimum bactericidal concentration is 60%-90% solutions in water by volume.

Ethyl alcohol, at concentrations of 60%-80%, is a potent virucidal agent inactivating all of the lipophilic viruses (e.g., herpes, vaccinia, and influenza virus) and many hydrophilic viruses (e.g., adenovirus, enterovirus, rhinovirus, and rotaviruses but not hepatitis A virus (HAV) 58 or poliovirus 49).

Isopropyl alcohol is not active against the nonlipid enteroviruses but is fully active against the lipid viruses. Studies also have demonstrated the ability of ethyl and isopropyl alcohol to inactivate the hepatitis B virus(HBV) and the herpes virus, and ethyl alcohol to inactivate human immunodeficiency virus (HIV), rotavirus, echovirus, and astrovirus.

In summary, at concentrations greater than 60 percent, alcohol effectively kills germs on your hands, electronics, and household surfaces. Microbes including bacteria, viruses, and fungi are susceptible to ethyl alcohol's germicidal effects. This includes the new coronavirus that causes the respiratory disease COVID-19.

However, despite the known benefits of such alcohol uses in connection with sanitizers and sanitizing wipes there are also known shortcomings of alcohols, and specifically alcohols effect on electrical equipment and electronic devices. Namely, such alcohols damage the shellac mountings of lensed instruments, tend to swell and harden rubber and certain plastic tubing after prolonged and repeated use, bleach rubber and plastic tiles and damage tonometer tips (by deterioration of the glue) after the equivalent of 1 working year of routine use. Additionally, alcohols are flammable and consequently must be stored in a cool, well-ventilated area. They also evaporate rapidly. Conversely, water is also damaging to electronics, and as a result one does not want a long evaporation time either.

What is needed, therefore, is a sanitizer and/or sanitizing wipe that is effective in cleaning electronic devices in a manner that effectively kills bacteria and viruses while not damaging the electronic device itself.

### BRIEF SUMMARY OF THE INVENTION

The present invention provides users with non-corrosive sanitizing wipes capable of being applied safely to cell phones and other electronic devices, and, in one embodiment, allows users to pull individual wipes from a resealable package, offering a simple way to acquire a single wipe for cleaning. The invention ensures the applied surface is fully sanitized of germs, bacteria, and viruses without causing damage to a cell phone screen and other similar devices, and functions as a wipe for shopping cart handles and numerous other high-traffic public surfaces. The invention also utilizes a packaging having a solid cover to prevent exposure to the air that may dry out the wipes while stored prior to use.

Each wipe is comprised of a non-scratching material saturated with an alcohol-based sanitizer. A plurality of wipes may be stored in a resealable container to prevent the wipes from drying out. Users may pull individual wipes out of the package for application. The package is resealed, and the wipe is applied to the desired surface. The non-scratching material ensures no damage is done to the applied surface. The sanitizing alcohol is designed to eradicate germs, viruses, and bacteria. Each package can be stored in a purse, pocket, backpack, etc. for convenient access. The wipes may also be provided in single use containers. Additionally, the sanitizing alcohol may be provided in the form of a spray to be used with reusable fabrics.

### BRIEF DESCRIPTION OF THE DRAWINGS

The present invention is described with reference to the accompanying drawings. In the drawings, like reference numbers indicate identical or functionally similar elements. The drawings are not drawn to scale.

FIG. 1A is an top perspective view of sample packaging.

FIG. 1B illustrates a sample wipe.

FIG. 2A illustrates the device being used to clean the back of a phone.

FIG. 2B illustrates the device being used to clean the front of a phone.

### DETAILED DESCRIPTION OF THE INVENTION

The present invention will now be described more fully in detail with reference to the accompanying drawings, in which the preferred embodiments of the invention are shown. This invention should not, however, be construed as limited to the embodiments set forth herein; rather, they are provided so that this disclosure will be complete and will fully convey the scope of the invention to those skilled in the art.

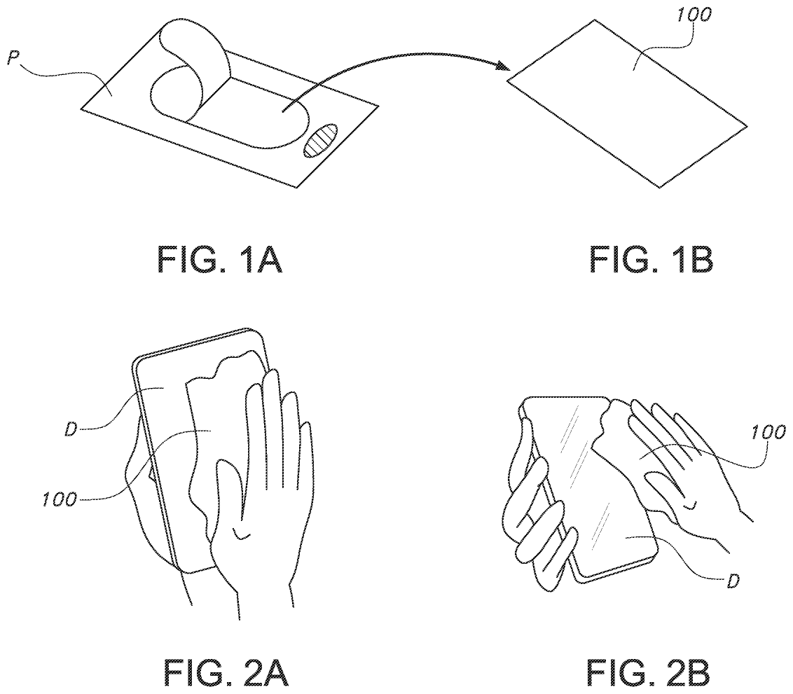

FIGS. 1A-2B illustrate a first embodiment of a sanitizing wipe 100 adapted to be used with electronic devices such as cellular phones that include a wipe that is saturated with a sanitizing solution. The wipe is made of a non-scratching and absorbent material that is capable of holding and distributing the sanitizing solution without damaging the electronic device D. The sanitizing solution is comprised of water and either ethyl alcohol or isopropyl alcohol at varying levels depending on the specific intended use of the wipe, i.e. what types of bacteria and/or viruses the wipe is adapted to kill.

The wipe 100 may be formed from any of a variety of materials as is well known in the art. Typically, however, the wipe includes a fibrous web that contains absorbent fibers. The wipe is made from biopolymer materials such as cotton, hemp, bamboo, abaca, wool, silk, ramie, jute, wood pulp, viscose rayon. The wipe may be made from 100% of a particular biopolymer, or it may be a combination of different biopolymer materials. Regardless of the choice of materials the basis weight of the wipe is typically from about 20 grams per square meter (“gsm”) to about 200 gsm, and in some embodiments, between about 35 to about 100 gsm. Products on the lower end of that basis weight range may be also be used for “light duty” wipes, while higher basis weight products may be better adapted for use as industrial purposes.

The wipe may assume a variety of shapes, including but not limited to, generally circular, oval, square, or rectangular. Each individual wipe may be arranged in a folded configuration, using conventional techniques, and stacked one on top of the other to provide a stack of wipes. The wipe may have an unfolded length of from about 5.0 to about 20.0 centimeters, and in some embodiments, but may vary in size depending on the intended purpose. For example, the wipes may likewise have an unfolded width of from about 2.0 to about 80.0 centimeters. The stack of folded wipes may be placed in the interior of a container P, such as a plastic tub or bag, to provide a package of wipes for eventual sale to the consumer. Alternatively, the wipes may include a continuous strip of material which has perforations between each wipe and which may be arranged into a roll for dispensing. Such a wipe does not scratch the surface or otherwise damage the electronic devices and is biodegradable, compostable, and flushable. Each wipe is intended for a single use.

The sanitizing solution includes between 5% to 70% by volume of water and between 30% and 95% by volume of alcohol. However, the preferred embodiments include a relatively high percentage of alcohol so that the solution evaporates quickly on the device surface, thereby minimizing and damage done by the solution to the device. As such, while a number of the following formulas may be used, those that feature an alcohol percentage of at least 70 and preferably 90 or higher, by volume, are preferred and recommended.

Following are a list of specific formulas for specific purposes, all percentages are by volume:
30% Ethyl Alcohol & 70% Water: Pneumonia;
40% Ethyl Or Isopropyl Alcohol & 60% Water: Salmonella, E. coli, Staphylococcus aureus;
Ethyl or Isopropyl As low as 60% & up to 80% & 40-20% Water: Staphylococcus, Streptococcus, Herpes, Flu, Adenovirus, Rhinovirus, Rotavirus, Hepatitis B, as well as all substances listed in the previous examples;
70% Ethyl & 30% Water: Cryptococcus neoformans, Blastomyces dermatitidis, Coccidioides immitis, and Histoplasma capsulatum;
70% Ethyl or Isopropyl Alcohol & 30% Water: Coronavirus, as well as all substances listed in the previous examples;
90% Ethyl Alcohol & 10% Water: IV, Bacteriocidal, Fungicidal, Virocidal, as well as all substances listed in the previous examples;
95% Ethyl Alcohol & 5% Water: Tuberculosis, as well as all substances listed in the previous examples.

Again, based on the preference to have a high alcohol percentage the formulas that include 90% to 95% Ethyl Alcohol are preferred, although lower percentages, e.g. 70%, may be suitable depending on the desired sanitizing strength of the solution, i.e. which types of virus or bacteria it needs to sanitize.

The amount of the sanitizing solution that is applied to each wipe is conventional and varies depending on the size of the wipe. Those skilled in the art are able to determine the proper amount of solution for the intended size of the wipe, however, for example, if the wipe is four inches in width and four inches in length with a thickness of 1/32 of an inch the amount of solution applied to the wipe is likely in the range of approximately 1 to 2 ounces. The sanitizing solution is applied to the wipes using conventional techniques.

The storage packaging, whether it be for single use packets containing only one wipe or multi-wipe package that contain a plurality of wipes, is comprised of conventional packaging materials that are known to hold moisture and prevent the solution from evaporating when not in use, for example polypropylene, polyethylene, polystyrene and the like.

In a second embodiment the sanitizing solution is provided in a conventional spray bottle and a reusable non-scratching cloth is provided for apply the solution to the electronic device. The sanitizing solution is identical to those listed in the first embodiment, and the reusable non-scratching cloth is made from a biopolymer solution.

It is understood that the embodiments described herein are merely illustrative of the present invention. Variations in the construction of the inventive sanitizing wipe may be contemplated by one skilled in the art without limiting the intended scope of the invention herein disclosed and as defined by the following claims.

What is claimed is:

1. A sanitizing wipe adapted to kill bacteria and viruses on electronic devices, the sanitizing wipe comprising:
a wipe that is made from biopolymer materials; and
a sanitizing solution that includes at least approximately 70% by volume of alcohol is applied to the wipe.

2. The sanitizing wipe of claim 1, the sanitizing solution including at least approximately 90% by volume of alcohol.

3. The sanitizing wipe of claim 1, the sanitizing solution including at least approximately 95% by volume of alcohol.

4. The sanitizing wipe of claim 3, the sanitizing solution including approximately 95% by volume of alcohol and 5% by volume of water.

5. The sanitizing wipe of claim 1, the wipe being made from a material that includes cotton.

6. The sanitizing wipe of claim 1, the wipe being made from a material that includes wood pulp.

7. The sanitizing wipe of claim 1, wherein the alcohol is an ethyl alcohol.

8. The sanitizing wipe of claim 1, wherein the alcohol is an isopropyl alcohol.

9. A container of sanitizing wipes, the sanitizing wipes adapted to kill bacteria and/or viruses on electronic devices, the container of sanitizing wipes comprising:
a storage packaging made of a material that holds moisture;
at least one wipe;
a sanitizing solution that includes at least approximately 90% by volume of alcohol is applied to the wipe.

10. The container of claim 9, wherein the at least one wipe includes only one wipe.

11. The container of claim 9, wherein the at least one wipe includes a plurality of wipes.

12. The container of claim 9, wherein the sanitizing solution includes at least approximately 95% by volume of alcohol.

13. The container of claim 12, wherein the alcohol is either an ethyl alcohol or an isopropyl alcohol.
